SEQUENCE LISTING (9 sequences)

SEQ ID NO: 1 - protein, 19 aa, Homo sapiens
  MOD_RES (10)..(10): phosphorylation
DRKMEVEELSPLALGRFSL

SEQ ID NO: 2 - protein, 13 aa, Homo sapiens
  MOD_RES (7)..(7): phosphorylation
MEVEELSPLALGR

SEQ ID NO: 3 - protein, 566 aa, Homo sapiens
  MOD_RES (235)..(235): phosphorylation
MEVPQPEPAPGSALSPAGVCGGAQRPGHLPGLLLGSHGLLGSPVRAAASSPVTTLTQTMHDLAGLGSRSRLTHLSLSRRASESSLSSESSESSDAGLCMDSPSPMDPHMAEQTFEQAIQAASRIIRNEQFAIRRFQSMPVRLLGHSPVLRNITNSQAPDGRRKSEAGSGAASSSGEDKENDGFVFKMPWKPTHPSSTHALAEWASRREAFAQRPSSAPDLMCLSPDRKMEVEELSPLALGRFSLTPAEGDTEEDDGFVDILESDLKDDDAVPPGMESLISAPLVKTLEKEEEKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 4 - protein, 539 aa, Homo sapiens
  MOD_RES (208)..(208): phosphorylation
MEVPQPEPAPGSALSPAGVCGGAQRPGHLPGLLLGSHGLLGSPVRAAASSPVTTLTQTMHDLAGLGSETPKSQVGTLLFRSRSRLTHLSLSRRASESSLSSESSESSDAGLCMDSPSPMDPHMAEQTFEQAIQAASRIIRNEQFAIRRFQSMPDGFVFKMPWKPTHPSSTHALAEWASRREAFAQRPSSAPDLMCLSPDRKMEVEELSPLALGRFSLTPAEGDTEEDDGFVDILESDLKDDDAVPPGMESLISAPLVKTLEKEEEKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 5 - protein, 580 aa, Homo sapiens
  MOD_ RES (249)..(249): phosphorylation
MEVPQPEPAPGSALSPAGVCGGAQRPGHLPGLLLGSHGLLGSPVRAAASSPVTTLTQTMHDLAGLGSETPKSQVGTLLFRSRSRLTHLSLSRRASESSLSSESSESSDAGLCMDSPSPMDPHMAEQTFEQAIQAASRIIRNEQFAIRRFQSMPVRLLGHSPVLRNITNSQAPDGRRKSEAGSGAASSSGEDKENDGFVFKMPWKPTHPSSTHALAEWASRREAFAQRPSSAPDLMCLSPDRKMEVEELSPLALGRFSLTPAEGDTEEDDGFVDILESDLKDDDAVPPGMESLISAPLVKTLEKEEEKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 6 - protein, 601 aa, Homo sapiens
  MOD_RES (259)..(259): phosphorylation
MEVPQPEPAPGSALSPAGVCGGAQRPGHLPGLLLGSHGLLGSPVRAAASSPVTTLTQTMHDLAGLGSRSRLTHLSLSRRASESSLSSESSESSDAGLCMDSPSPMDPHMAEQTFEQAIQAASRIIRNEQFAIRRFQSMPVRLLGHSPVLRNITNSQAPDGRRKSEAGSGAASSSGEDKENVRFWKAGVGALREEEGACWGGSLACEDPPLPSWLQDGFVFKMPWKPTHPSSTHALAEWASRREAFAQRPSSAPDLMCLSPDRKMEVEELSPLALGRFSLTPAEGDTEEDDGFVDILESDLKDDDAVPPGMESLISAPLVKTLEKEEEKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 7 - protein, 588 aa, Homo sapiens
  MOD_RES (284)..(284): phosphorylation
MEVPQPEPAPGSALSPAGVCGGAQRPGHLPGLLLGSHGLLGSPVRAAASSPVTTLTQTMHDLAGLGSETPKSQVGTLLFRSRSRLTHLSLSRRASESSLSSESSESSDAGLCMDSPSPMDPHMAEQTFEQAIQAASRIIRNEQFAIRRFQSMPVRLLGHSPVLRNITNSQAPDGRRKSEAGSGAASSSGEDKENVRFWKAGVGALREEEGACWGGSLACEDPPLPSWLQDGFVFKMPWKPTHPSSTHALAEWASRREAFAQRPSSAPDLMCLSPDRKMEVEELSPLALGRFSLTPAEGDTEEDDGFVDILESDLKDLVMYSKCQRLFRSPSMPCSVIRPILKRLERPQDRDTPVQNKRRRSVTPPEEQQEAEEPKARVLRSKSLCHDEIENLLDSDHRELIGDYSKAFLLQTVDGKHQDLKYISPETMVALLTGKFSNIVDKFVIVDCRYPYEYEGGHIKTAVNLPLERDAESFLLKSPIAPCSLDKRVILIFHCEFSSERGPRMCRFIRERDRAVNDYPSLYYPEMYILKGGYKEFFPQHPNFCEPQDYRPMNHEAFKDELKTFRLKTRSWAGERSRRELCSRLQDQ

SEQ ID NO: 8 - protein, 580 aa, Homo sapiens; identical to SEQ ID NO: 5

SEQ ID NO: 9 - protein, 13 aa, Homo sapiens
MEVEELSPLALGR

(19) United States
(12) Patent Application Publication
(10) Pub. No.: US 2008/0219987 A1
(43) Pub. Date: Sep. 11, 2008

(54) Novel Phosphorylated Phosphatase Cdc25b Sequences, Antibodies Directed Against Said Sequences and Uses Thereof

(76) Inventors: Bernard Ducommun, Belberaud (FR); Martine Cazales, Balma (FR); Bernard Monsarrat, Albi (FR)

Correspondence Address: YOUNG & THOMPSON, 209 Madison Street, Suite 500, ALEXANDRIA, VA, 22314, US

(21) Appl. No.: 11/660,703

(22) PCT Filed: Aug. 23, 2005
(86) PCT No.: PCT/FR05/02126

§ 371 Date: Dec. 10, 2007

(30) Foreign Application Priority Data
Aug. 25, 2004 (FR) 0409080

(51) Int. Cl. A61K 39/395; C07K 4/12; C07K 7/06; C07K 7/08; C07K 16/18; C12P 21/04; A61K 38/03; A61K 38/08; A61K 38/10; G01N 33/00

(52) U.S. Cl. 424/139.1; 530/328; 530/327; 530/326; 530/387.9; 435/70.21; 514/15; 514/14; 514/13; 435/7.23

(57) ABSTRACT

A peptide sequence includes a fragment of at least 10 amino acids derived from the following SEQ ID NO:1: DRKMEVEELSₚPLALGRFSL SEQ ID NO:1 wherein the serine residue in position 10 is phosphorylated, the above-mentioned fragment containing the phosphorylated serine residue. A polyclonal or monoclonal antibody capable of identifying a peptide sequence as defined above is also disclosed.

A subject of the present invention is novel phosphorylated CDC25CB phosphatase sequences as well as polyclonal or monoclonal antibodies directed against these sequences. A subject of the present invention is also the use of these novel phosphorylated sequences in particular for the implementation of an in vitro method for the screening of compounds inhibiting cell mitosis, namely compounds inhibiting the cells' entry into mitosis or the progression of the mitosis.

The mechanisms which control cell division involve numerous agents the activities of which are regulated by phosphorylation and dephosphorylation reactions, involving kinases and phosphatases. Deregulations of these mechanisms have been identified in numerous cancers. Their identification and their characterization are now opening up new perspectives for the diagnosis and treatment of cancer.

CDC25B is a cell cycle regulatory phosphatase which is essential for controlling entry into mitosis and the progression of mitosis. It belongs to a family which comprises three members coded by different genes (CDC25A, B and C) in mammals. The CDC25B protein is expressed and active at the end of the G2 phase of the cell cycle (Baldin et al., 1997; Gabrielli et al., 1996). Its intracellular localization is regulated by NES and NLS sequences (Davezac et al., 2000) and by its interaction with the 14-3-3 proteins (Mils et al., 2000; Forrest et al., 2001). It has been suggested that CDC25B can act as a “starter” of early mitotic events (Nilsson et al., 2000). It could play a role in the initial activation of a population of CDC2/cyclin B at the level of the centrosome before its nuclear translocation (Kumagai et al., 1992; Hoffmann et al., 1993). CDC25B activates the CDK/cyclin complexes in order to allow the architectural and biochemical changes which are necessary in order to allow the cell division process. Its activity is regulated by the variations in its expression, by its combination with regulatory partners and by phosphorylation events. Thus, a signalling cascade leads to the modulation of the catalytic activity of CDC25B participating in the regulation of entry into mitosis.

One of the purposes of the invention is to provide a novel phosphorylated CDC25B phophosphatase sequence, as well as a novel antibody directed against the phosphorylated epitope of the phosphorylated CDC25B phophosphatase.

One of the purposes of the present invention is to provide an in vitro method for the screening of compounds inhibiting cell mitosis, in particular of entry into mitosis, said inhibitory compounds being able to be in particular used within the framework of an anticancer therapy.

The present invention relates to a peptide sequence characterized in that it comprises or is constituted by a fragment of at least approximately 10 amino acids originating from the following sequence SEQ ID NO: 1:

| | |
|---|---|
| DRKMEVEELSₚPLALGRFSL | SEQ ID NO: 1 |

in which the serine residue in position 10 is phosphorylated,

said abovementioned fragment containing said phosphorylated serine residue.

The expression “phosphorylated residue” denotes an amino acid carrying a phosphate group.

According to an advantageous embodiment, the present invention relates to a peptide sequence as defined above, characterized in that it comprises or is constituted by the following sequence SEQ ID NO: 2:

| | |
|---|---|
| MEVEELSₚPLALGR | SEQ ID NO: 2 |

in which the serine residue in position 7 is phosphorylated.

The sequence SEQ ID NO: 2 corresponds to a fragment of the abovementioned sequence SEQ ID NO: 1. More precisely, it corresponds to the fragment of SEQ ID NO: 1 delimited by the amino acid in position 4 to the amino acid in position 16.

The present invention also relates to a peptide sequence as defined above, characterized in that it comprises or is constituted by one of the following sequences:
the sequence SEQ ID NO: 3, representing the CDC25B1 splice variant of the CDC25B phosphatase protein of human origin, the serine residue of which in position 235 is phosphorylated,
the sequence SEQ ID NO: 4, representing a CDC25B2 splice variant of the CDC25B phosphatase protein of human origin, the serine residue of which in position 208 is phosphorylated,
the sequence SEQ ID NO: 5, representing a CDC25B3 splice variant of the CDC25B phosphatase protein of human origin, the serine residue of which in position 249 is phosphorylated,
the sequence SEQ ID NO: 6, representing a CDC25B4 splice variant of the CDC25B phosphatase protein of human origin, the serine residue of which in position 259 is phosphorylated,
the sequence SEQ ID NO: 7, representing a CDC25B5 splice variant of the CDC25B phosphatase protein of human origin, the serine residue of which in position 284 is phosphorylated.

The present invention also relates to a polyclonal or monoclonal antibody capable of recognizing a peptide sequence as defined above, providing that said antibody does not recognize the sequence SEQ ID NO: 8 in which the serine residue in position 249 is not phosphorylated.

The sequence SEQ ID NO: 8 corresponds to the non-phosphorylated CDC25B protein sequence.

An advantageous polyclonal antibody of the invention is characterized in that it is capable of recognizing the sequence SEQ ID NO: 2 as defined above, providing that said antibody does not recognize the sequence SEQ ID NO: 8 in which the serine residue in position 249 is not phosphorylated.

Such an antibody directed against the phosphorylated epitope of sequence SEQ ID NO: 2 is produced by immunizing rabbits with said epitope.

More precisely, said epitope is covalently coupled with a carrier protein such as hemocyanin, BSA or ovalbumin. The rabbits are then immunized over 3 months (4 injections in total) and the final bloodletting allows the recovery of approximately 50 ml of serum. The serum is then doubly affinity-purified on a phosphorylated peptide column then on a non-phosphorylated peptide column.

The present invention also relates to a process for the preparation of a monoclonal antibody as defined above, in particular directed against the peptide sequence SEQ ID NO: 2 as defined above, characterized in that it results from the selection of a hybridoma secreting an antibody directed against the peptide sequence as defined above or from the selection from an expression bank of a complementary DNA coding for all or part of an antibody.

The present invention also relates to a process for the preparation of a monoclonal antibody as defined above, in particular directed against the peptide sequence SEQ ID NO: 2 as defined above, characterized in that it comprises the following stages:
the fusion between myelomas from an immunized animal, in particular a non-human animal immunized beforehand, by injection of the peptide sequence as defined above and splenocytes from an animal, in particular a non-human animal, in order to obtain hybridomas,
the culture of the hybridomas thus obtained,
the recovery and purification by cloning of a hybridoma, chosen from those obtained in the preceding stage and secreting an antibody directed against the peptide sequence as defined above.

The animal used for the immunization stage is in particular a mouse.

The myelomas used for the fusion originate in particular from a mouse.

The splenocytes used for the fusion originate from an animal of the same species as that from which the myelomas originate, namely in particular from a mouse.

Hybridomas are chosen which secrete the antibodies against the peptide sequence SEQ ID NO: 2 on the basis of the production of antibodies capable of recognizing in an ELISA test the phosphorylated peptide used for the immunization but not the non-phosphorylated peptide.

The present invention also relates to a process for the preparation of a monoclonal antibody as defined above, in particular directed against the peptide sequence SEQ ID NO: 2 as defined above, characterized in that it comprises a stage of selection from an expression bank of a cDNA coding for all or part of an antibody.

The antibodies are selected against the peptide sequence SEQ ID NO: 2 on the basis of their ability to recognize in an ELISA test, by protein transfer (Western Blot) or by any other appropriate method, the phosphorylated peptide used for the immunization, but not the non-phosphorylated peptide.

The present invention also relates to a pharmaceutical composition characterized in that it contains as active ingredient a peptide sequence as defined above or an antibody as defined above, in combination with a pharmaceutically acceptable vector.

The present invention also relates to the use of the peptide sequence as defined above, or of an antibody as defined above, for the preparation of a medicament intended for the treatment of hyperproliferative diseases such as cancers.

The present invention also relates to the use of an antibody as defined above, for the implementation of a method for the in vitro detection of mitotic cells, expressing a protein sequence as defined above, from cells in culture or from sections of healthy or tumorous tissues.

The cells in culture are fixed, permeabilized then incubated in the presence of the antibody. The visualization of the antibody is carried out by use of a secondary antibody carrying a fluorochrome (the observation is then carried out with a fluorescence microscope) or of a molecule allowing the hydrolysis of a substrate and the production of a colour reaction observable under visible light. A similar experimental strategy can be used on tissue sections.

The present invention also relates to the use of an antibody as defined above, for the implementation of a method for the in vitro detection of the overexpression of a protein sequence as defined above, in cells in culture or sections of healthy or tumorous tissues, in particular in sections of breast, lung or pancreatic tumours.

Demonstration of the overexpression of CDC25B is based on the same experimental strategy as described above for the method for the detection of mitotic cells. It can also be carried out by protein transfer (Western Blot) or by any other method capable of quantifying a protein of interest on total protein extracts prepared from healthy or tumorous cells.

The present invention also relates to an in vitro method for the screening of compounds inhibiting mitosis, namely the entry into mitosis of the cells or the progression of the mitosis, said inhibitory compounds being able to be in particular used within the framework of an anticancer therapy, characterized in that it comprises:
bringing said compound together with cells, and
selection of the inhibitory compounds by detection of the absence of binding of the antibody as defined above, said antibody being capable of recognizing a peptide sequence as defined above, by an appropriate method, in particular using ELISA, protein transfer (Western Blot) or indirect immunofluorescence techniques.

### DESCRIPTION OF THE FIGURES

FIGS. 1A, 1B, 1C, 1D and 1E represent the detection of the phosphorylated form of CDC25B at serine 249 by immunofluorescence at different cell stages. FIG. 1A corresponds to the interphase; FIG. 1B corresponds to the prophase; FIG. 1C corresponds to the metaphase; FIG. 1D corresponds to the anaphase/telophase; and FIG. 1E corresponds to the G1 phase.

FIG. 2 represents the detection of the phosphorylated form of CDC25B at serine 249 by protein transfer (Western Blot).

Column 1 corresponds to the CDC25B protein purified alone; column 2 corresponds to the CDC25B protein purified with the phosphorylated peptide; column 3 corresponds to the CDC25B protein purified with the non-phosphorylated peptide; and column 4 corresponds to the treatment of CDC25B with lambda phosphatase.

### METHODS AND RESULTS

CDC25B Phosphatase is Phosphorylated at Serine 249

Mass spectrometry analysis carried out on CDC25B made it possible to detect the phosphorylation of the serine residue in position 249.

The CDC25B protein was purified then digested by trypsin. MS/MS mass spectrometry analysis revealed the presence of a monophosphorylated peptide. The fragmentation of this peptide allowed the identification of the phosphate group at serine 249.

Production of Antibodies Against the Phosphorylated CDC25B Protein at the Amino Acid Serine 249

The peptide of sequence MEVEELS(p)PLALGR (SEQ ID NO: 2), where (p) denotes the phosphorylated serine, was used for the immunization of rabbits. After sacrificing the animals, the serum was purified by chromatography in two stages: the first on a column of phosphorylated peptide in order to retain the specific antibodies, then the second on a column of the same non-phosphorylated peptide of sequence MEVEELSPLALGR (SEQ ID NO: 9), so as to purify the specific antibodies of phosphorylated form in the eluate. Recognition of the phosphorylated peptide by the antibodies was validated in an ELISA test. Hereafter, these antibodies are denoted by the name anti-S249P.

The Anti-S249P Antibodies Recognize the CDC25B Protein in Cells in Mitosis.

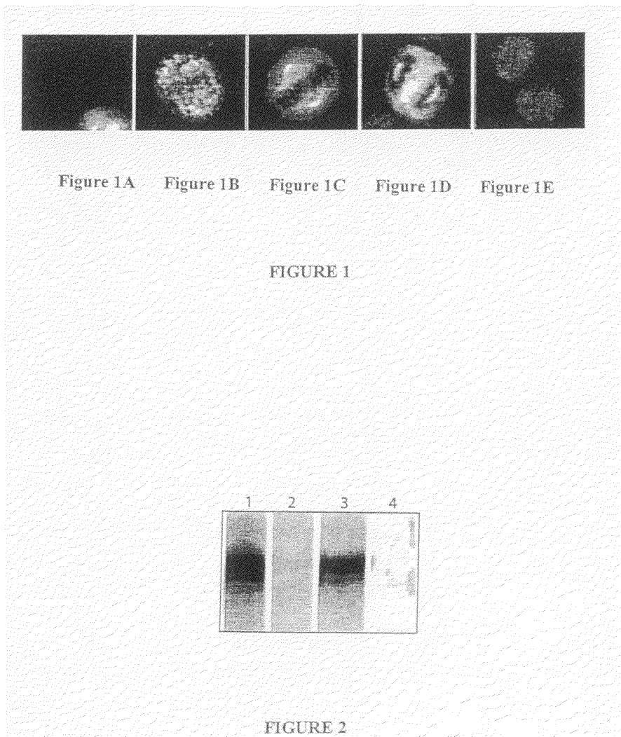

A) HeLa cells were fixed and used to carry out immunofluorescence analysis with these antibodies. The cells were also stained with 4′-6-diamino-2-phenylindole (DAPI) in order to locate the nucleus. The images shown in FIG. 1 are representative of observations carried out on a large number of cells. They indicate that the phosphorylated CDC25B protein at serine 249 (SEQ ID NO: 2) is accumulated very abundantly in the cells in mitosis, in particular at the level of the spindle poles. This marking is abolished when a competition is carried out with the test with the phosphorylated peptide having served for the immunization but not with the non-phosphorylated peptide (MEVEELSPLALGR) nor with an unconnected phosphorylated peptide.

B) The CDC25B protein was purified from cells in culture then analyzed by protein transfer (Western Blot) with the antibodies against the phosphorylated CDC25B phophosphatase at the level of serine 249. As shown in FIG. 2, the CDC25B protein is phosphorylated in vivo on this site. The treatment with lambda phosphatase abolishes this phosphorylation. The detection is eliminated by competition with the phosphorylated peptide having served for the immunization.

These observations validate the use of these antibodies for the detection of the phosphorylated CDC25B protein in mitotic cells.

### FIELDS OF APPLICATION

A—Mitotic Cell Marker:

Detection of the phosphorylation of the phosphorylated form of CDC25B makes it possible to detect the presence of mitotic cells on cells in culture or on sections of tumours.

The phosphorylated form of CDC25B at serine 249 is present in the cell in mitosis. Its localization is nuclear in prophase, then it is concentrated in metaphase on the two mitotic hemispindles before invading at equatorial level in telophase, then disappearing at the end of mitosis.

The polyclonal or monoclonal antibodies directed against this phosphorylated form of CDC25B consequently allow the detection of any mitotic cell expressing CDC25B phosphatase.

This detection of mitotic cells can be carried out on fixed cells in culture or on sections of healthy or tumorous tissues, using the indirect immunofluorescence or immunocytochemical techniques.

The detection of mitotic cells by this method is thus a novel tool at the disposal of cytologists and anatomical pathologists.

B—Assisting Therapeutic Decisions:

Taking into account the levels of expression of CDC25B in tumours is of major benefit in the choice of therapeutic decision (use or non-use of drugs targeting the CDC25B protein). Detection of the phosphorylated form of CDC25B at serine 249 is of major benefit in this application.

The expression of CDC25B is variable depending on the tissues. It has been shown that tumours (breast, lung, pancreatic etc.) overexpress this protein. In the case of pancreatic tumours, the tumour growth is dependent on the expression and function of CDC25B (Guo et al., 2004).

New pharmacological agents capable of targeting CDC25B are currently being developed by the industry (Prevost et al., 2003). It is therefore essential to take into account the level of expression of CDC25B phophosphatase in order to determine the relevance of the use of such a treatment.

The use of antibodies directed against the phosphorylated form of CDC25B at serine 249 makes it possible to visualize the mitotic (and probably active) form of this phosphatase. Its detection (by immunocytochemistry) in biopsies or on pieces of tissue following surgical exeresis provides elements of information of a kind to direct the therapeutic choice by optionally providing, if CDC25B is overexpressed, the indication of a use of phosphatase CDC25 inhibitors.

C—Setting Up of High-Throughput Screening:

Research into molecules interfering with the cell cycle can use the antibodies according to the invention in order to evaluate the inhibition of entry into mitosis, Active research into molecules inhibiting progression in the cell cycle is currently being carried out by numerous pharmaceutical groups.

The demonstration of a mitotic cell marker represents a tool of choice for exploring, in a simple manner and in high-throughput screening, the ability of molecules to inhibit entry into and progression in mitosis.

The antibodies against the phosphorylated form of CDC25B can meet this need. They can thus be used in immunocytochemistry, in flow cytometry or by any other suitable method making it possible to detect the phosphorylated CDC25B protein.

### REFERENCES

Baldin, V., Cans, C., Superti-Furga, G. & Ducommun, B (1997) Alternative splicing of the human CDC25B tyrosine phosphatase. Possible implications for growth control? Oncogene, 14, 2485-2495,
Davezac, N. et al. (2000) Regulation of CDC25B phosphatases subcellular localization, Oncogene, 19, 2179-85,
Forrest A. & Gabrielli, B. (2001) Cdc25B activity is regulated by 14-3-3, Oncogene, 20, 4393-401,
Gabrielli, B. G. et al. (1996) Cytoplasmic accumulation of CDC25B phosphatase in mitosis triggers centrosomal microtubule nucleation in HeLa cells, J. Cell. Science, 109, 1081-1093,
Guo et al. (2004) Expression and functional significance of CDC25B in human pancreatic ductal adenocarcinoma, Oncogene, 23, 71-81,
Hoffmann, I., Clarke, P., Marcote, M. J., Karsenti, E. & Draetta, G. (1993) Phosphorylation and activation of human cdc25-C by cdc2-cyclin. B and its involvement in the self amplification of MPF at mitosis, EMBO J, 12, 53-63,
Kumagai, A. & Dunphy, W. (1992) Regulation of the cdc25 protein during the cell cycle in Xenopus extracts, Cell, 70, 139-151,
Mils, V. et al. (2000) Specific interaction between 14.3.3 isoforms and the human CDC25B phosphatase, Oncogene, 19, 1257-1265,
Nilsson, I. & Hoffmann, I. (2000) Cell cycle regulation by the Cdc25 phosphatase family, Prog Cell Cycle Res, 4, 107-14,
Prevost, G. et al. (2003) Inhibitors of the CDC25 phosphatases in “Progress in cell cycle research” Ed. Meijer, L., Jézéquel, A., Roberge, M., vol. 5, 225-234.

1-12. (canceled)

13. Peptide sequence comprising or constituted by a fragment of at least (approximately) 10 amino acids originating from the following sequence SEQ ID NO: 1:
DRKMEVEELS_{P}PLALGRFSL SEQ ID NO: 1
in which the serine residue in position 10 is phosphorylated,
said abovementioned fragment containing said phosphorylated serine residue.

14. Peptide sequence according to claim 13, comprising or constituted by the following sequence SEQ ID NO: 2:
MEVEELS_{P}PLALGR SEQ ID NO: 2
in which the serine residue in position 7 is phosphorylated.

15. Peptide sequence according to claim 13, comprising or constituted by one of the following sequences:
the sequence SEQ ID NO: 3, representing the CDC25B1 splice variant of the CDC25B phosphatase protein of human origin, the serine residue of which in position 235 is phosphorylated,
the sequence SEQ ID NO: 4, representing a CDC25B2 splice variant of the CDC25B phosphatase protein of human origin, the serine residue of which in position 208 is phosphorylated,
the sequence SEQ ID NO: 5, representing a CDC25B3 splice variant of the CDC25B phosphatase protein of human origin, the serine residue of which in position 249 is phosphorylated,
the sequence SEQ ID NO: 6, representing a CDC25B4 splice variant of the CDC25B phosphatase protein of human origin, the serine residue of which in position 259 is phosphorylated,
the sequence SEQ ID NO: 7, representing a CDC25B5 splice variant of the CDC25B phosphatase protein of human origin, the serine residue of which in position 284 is phosphorylated.

16. Polyclonal or monoclonal antibody capable of recognizing a peptide sequence according to claim 13, providing that said antibody does not recognize the sequence SEQ ID NO: 8 in which the serine residue in position 249 is not phosphorylated.

17. Polyclonal antibody capable of recognizing the sequence SEQ ID NO: 2 as defined in claim 14, providing that said antibody does not recognize the sequence SEQ ID NO: 8 in which the serine residue in position 249 is not phosphorylated.

18. Process for the preparation of a monoclonal antibody directed against the peptide sequence SEQ ID NO: 2 as defined in claim 14, (characterized in that it) comprising the following stages:
the fusion between myelomas from an immunized animal, a non-human animal immunized beforehand, by injection of the peptide sequence and of splenocytes from an animal, in order to obtain hybridomas,
the culture of the hybridomas thus obtained,
the recovery and purification by cloning of a hybridoma, chosen from those obtained in the preceding stage and secreting an antibody directed against the peptide sequence.

19. Process for the preparation of a monoclonal antibody, directed against the peptide sequence SEQ ID NO: 2 as defined in claim 14, comprising a stage of selection from an expression bank of a cDNA coding for all or part of an antibody, said antibody being selected on the basis of its ability to recognize in an ELISA test, by protein transfer or by any other appropriate method, the phosphorylated peptide used for the immunization, but not the non-phosphorylated peptide.

20. Pharmaceutical composition containing as active ingredient a peptide sequence according to claim 13, in combination with a pharmaceutically acceptable vector.

21. Method of treatment of hyperproliferative diseases comprising the administration of the peptide sequence according to claim 13, to a patient in need thereof.

22. Method for the in vitro detection of mitotic cells expressing a protein sequence according to claim 13, from cells in culture or from sections of healthy or tumorous tissues by an antibody capable of recognizing the peptide sequence, providing that said antibody does not recognize the sequence SEQ ID NO: 8 in which the serine residue in position 249 is not phosphorylated.

23. Method for the in vitro detection of the overexpression of a protein sequence according to claim 13, in cells in culture or sections of healthy or tumorous tissues, selected from the group consisting of sections of breast, lung or pancreatic tumours by an antibody capable of recognizing the peptide sequence, providing that said antibody does not recognize the sequence SEQ ID NO: 8 in which the serine residue in position 249 is not phosphorylated.

24. Method for the in vitro screening of inhibitory compounds of cell mitosis, namely of the cells' entry into mitosis or the progression of the mitosis, said inhibitory compounds being able to be used within the framework of an anticancer therapy, comprising:
bringing said compound together with cells, and
selection of the inhibitory compounds by detection of the absence of binding of the antibody according to claim 16, said antibody being capable of recognizing a peptide sequence, by an appropriate method selected from the group consisting of ELISA, transfer protein or indirect immunofluorescence techniques.

25. Pharmaceutical composition containing as active ingredient an antibody according to claim 16, in combination with a pharmaceutically acceptable vector.

26. Method of treatment of hyperproliferative diseases comprising the administration of an antibody according to claim 16 to a patient in need thereof.
